(19) United States
(12) Patent Application Publication
(10) Pub. No.: US 2021/0219903 A1
(43) Pub. Date: Jul. 22, 2021

(54) CARDIO CHAIR FOR RECORDING 1-LEAD ECG

(71) Applicants: Aleksandr Ezhkov, Moscow (RU); Sergey Sadovskiy, Moscow (RU); Olga Sountsova, Moscow (RU); Vadim Brendel, Lobnya (RU)

(72) Inventors: Aleksandr Ezhkov, Moscow (RU); Sergey Sadovskiy, Moscow (RU); Olga Sountsova, Moscow (RU); Vadim Brendel, Lobnya (RU)

(21) Appl. No.: 17/136,061

(22) Filed: Dec. 29, 2020

Related U.S. Application Data
(63) Continuation of application No. PCT/RU2020/000149, filed on Mar. 19, 2020.

(30) Foreign Application Priority Data
Sep. 4, 2019 (RU) 2019127893

(51) Int. Cl. A61B 5/333; A61B 5/00; A61B 5/277; A61B 5/28

(52) U.S. Cl. CPC A61B 5/333; A61B 5/28; A61B 5/277; A61B 5/6887

(57) ABSTRACT

The technology relates to medical equipment, namely, to a medical chair for recording an electrocardiogram in the first lead («1-lead»), and can be used in medical institutions, pharmacies, public organizations and at home.

Chair for measuring electrocardiogram in the first lead containing: two dry capacitive contact electrodes, each is attached to the appropriate armrest; wherein electrodes are made in the form of handles and configured in such a way as to enable the user to hold onto them with their palms in a sitting position; wherein electrodes are adapted to connect them to the lead cable for connection with the electrocardiograph, wherein electrodes are isolated from the chair frame through a dielectric spacer, wherein the electrocardiograph holder is attached to the chair so that the electrocardiograph secured in the holder is at user eye level.

### CROSS-REFERENCE

The present application claims priority to Russian Utility Patent Application No. 2019127893, filed on Sep. 4, 2018, entitled “Medical chair for recording 1-lead ECG” the entirety of which is incorporated herein by reference and is a continuation of PCT Application No. PCT/RU2020/000149 filed on Mar. 19, 2020, entitled “Cardio chair for recording 1-lead ECG”.

### FIELD OF THE TECHNOLOGY

The technology relates to medical equipment, namely, to a medical chair for recording an electrocardiogram in the first lead («1-lead»), and can be used in medical institutions, pharmacies, public organizations and at home.

### BACKGROUND

The electrocardiograph is a device well-known in the state of art for recording electrical vibrations (changes in potential differences) resulting from the cardiac performance. The result of measurements of an electrocardiograph is an electrocardiogram (ECG), which is a set of deflections, segments and intervals that reflect the complex process of wave propagation in the heart. Medical ECG interpretation provides the following information:
heart rate,
physical condition of the heart,
presence of arrhythmias,
presence of acute or chronic myocardial damage,
presence of metabolic disorders in the heart muscle,
presence of electrical conductivity disruptions,
position of the electrical axis of the heart,
an electrocardiogram can be used to obtain information about certain vascular diseases not related to the heart.

The main structural elements of the electrocardiograph are electrode sensors and the recording device itself. Changes in the potential difference on the body surface that occur during the cardiac performance are recorded with the help of various ECG lead systems. Each lead records the potential difference that exists between two specific points of the heart's electric field where the electrodes are installed.

A person skilled in the art knows the following standard leads:

Lead I (denoted in this application by the term “1-lead”)—left hand (+) and right arm (−);

Lead II—left leg (+) and right arm (−);

Lead III—left leg (+) and left arm (−).

Interference may occur during the ECG recording, i.e. electrical vibrations coming not from a working heart, but from other sources. Muscle tremor may become one of the reasons. Muscle tremor (irregular frequent small fluctuations of various frequencies and amplitudes) occurs when a patient experiences muscle shivering due to small contractions of skeletal muscles. Remedies: ask the patient to relax, do not keep the limbs in the suspended position, putting them on a support.

According to the current ECG recording technique, you need to place the patient horizontally on his/her back, usually on a medical couch, and expose his/her wrists (enough to record an ECG in lead I), legs and chest. Then you need to apply electrodes to specific points, depending on the type of lead. For the 1-lead, as mentioned above, these points are located on the right and left arm. After that, you need to take readings.

However, for example, if the patient has severe shortness of breath and cannot lie down, the ECG is recorded in a sitting position. In this position on the couch or chair, the patient may feel very uncomfortable and find it difficult to relax. Accordingly, increased muscle tremor will cause interference in the ECG recording and affect the measurement accuracy.

In addition, the lying position is not always convenient in terms of the location and availability of furniture required for measuring the ECG in the office or room space.

Due to the listed disadvantages of the used ECG measurement technique, there is a need to develop a chair in which it is possible to measure the ECG in a relaxed state. Furthermore, it is highly preferable to develop a chair in which the ECG measurement can be carried out independently without the help of, for example, medical staff.

The prior art discloses a medical chair, described in USSR patent N 1473769, class A 61 G 15/00, 1989, containing a support frame with a seat, a backrest and a footrest with movement mechanisms. To set the optimal position of the patient, a profiled cam is used, which interacts with a roller fixed to a support frame.

However, the disadvantage of the known chair is the inability of independent recording of the electrocardiogram in the 1-lead by the patient.

The prior art also discloses various designs of chairs with built-in electrodes for measuring the potential difference on the surface of the user's body, for example, described in the following sources:

Capturing Electrocardiogram Signals from Chairs by Multiple Capacitively Coupled Unipolar Electrodes by Zhongjie HouOrcID et al. (Sensors 2018, 18 (9), 2835; https://doi.org/10.3390/s18092835),

Non-contact, non-obtrusive electrocardiography in clinical environments by Kunal Mankodiya et al. (5th International Conference on Pervasive Computing Technologies for Healthcare, PervasiveHealth 2011, Dublin, Ireland, May 23-26, 2011);

ECG Measurement on a chair without conductive contact by Ko Keun Kim et al. (IEEE Transactions on Biomedical Engineering 53(5):956-959⋅June 2006);

Advances in Modern Capacitive ECG Systems for Continuous Cardiovascular Monitoring by A. Schommartz et al. (Acta Polytechnica Vol. 51 No. 5/2011).

However, all described chairs use non-contact capacitive electrodes. For these electrodes, the biopotential signal amplitude depends on the distance of the patient's body area to the sensors. The signal obtained in this way is not formally a medical ECG, since a device with such electrodes does not comply with the international standard for electrocardiographs IEC 60601-2-25-2016. The signal received from such chairs is only suitable for detecting arrhythmia, whereas other pathologies of the cardiovascular system cannot be diagnosed with such a signal.

Furthermore, the described chairs use a non-standard lead from the back, while the ground electrode is capacitive and is not attached to the patient's leg, as it should be according to the standard, but is built into the seat. The signal from such a lead (from the back) is not one of the standard leads and, again, from a medical point of view, is not significant and cannot be interpreted according to standard medical techniques.

Moreover, the medical chairs known from the prior art do not contain a cardiograph and, therefore, the measurement cannot be carried out independently, which results in the inability to independently start the ECG recording, independently control and complete it.

The technical challenge is to create a medical chair for ECG measurement, which would make it possible to diagnose the most common heart diseases independently and with a high degree of reliability of the results.

### SUMMARY

The objective of the present technology is ensuring high reliability of ECG results for diagnosing the most common heart diseases.

The technical result is achieved through the use of a medical chair proposed in this application for recording/measuring an ECG in the 1-lead, i.e. from arms. Recording of an ECG in the 1-lead allows diagnosing arrhythmia, coronary heart disease, cardiac conduction diseases, atrial fibrillation and other heart diseases. The armrests of the proposed medical chair include dry capacitive contact electrodes made in the form of handles, on which the patient puts his/her palms during measurement. Electrode handles allow you to set the maximum contact area of the hands with dry capacitive contact electrodes for measuring an ECG in the 1-lead, thereby increasing the quality of the signal received from the specified type of electrodes. At the same time, when measuring an ECG, the proposed medical chair allows placing the patient's body, including arms and hands, in a natural, most comfortable and relaxed state. In this position, the influence of muscle tremor on the signal quality is minimal and excludes the appearance of interference during the ECG recording.

Thus, the technical result of the claimed technical solution is achieved due to the fact that the medical chair for recording/measuring 1-lead ECG contains two dry capacitive contact electrodes, each of which is attached to a corresponding armrest. These electrodes are made in the form of handles and are configured to provide the user with the ability to hold onto them with his/her palms in a sitting position. In this case, the electrodes are adapted with the possibility of connecting them with a lead cable for connection with an electrocardiograph.

In accordance with various exemplary and non-limiting embodiments the electrodes are cylinder shaped.

In accordance with various exemplary and non-limiting embodiments the electrodes are made in the form of stainless steel handles, to which a brass petal can be attached from the inside by electric resistance welding, while the lead cable wire is soldered to the petal.

In accordance with various exemplary and non-limiting embodiments of the technical electrodes are part of the armrest, while the electrodes are isolated from the chair frame through a dielectric spacer.

In accordance with various exemplary and non-limiting embodiments chair comprises an electrocardiograph holder.

In accordance with various exemplary and non-limiting embodiments the chair is equipped with a metal tube, to which the electrocardiograph holder is attached so that the electrocardiograph fixed in the holder is at eye level of the user. This provides easy access to the control of the electrocardiograph functions.

In accordance with various exemplary and non-limiting embodiments the chair consists of a frame, a seat, a seat back and two armrests, which can be made with the possibility of an ergonomic positioning of the patient in the chair in a sitting position, and the frame at least partially can be made of hollow tubes, which may hide cable wires.

In accordance with various exemplary and non-limiting embodiments the frame can be metal (made of metal) and have a terminal for connecting the ground conductor of the electrocardiograph.

In accordance with various exemplary and non-limiting embodiments the medical chair for recording 1-lead ECG comprises two dry capacitive contact electrodes, each of which is attached to a corresponding armrest, and a cardiograph holder. The electrodes are made in the form of handles and are configured to allow the user to hold onto them with the palms of their hands in a sitting position. The electrodes are connected by a lead cable to the cardiograph, which is fixed in the cardiograph holder. The cardiograph holder is designed to hold the cardiograph in a position convenient for the user.

### BRIEF DESCRIPTION OF THE DRAWINGS

FIG. 1 shows a front view of an example of the proposed technical solution.

FIG. 2 shows a top view of an example of the proposed technical solution.

FIG. 3 shows an exemplary design of the electrocardiograph holder.

### DETAILED DESCRIPTION

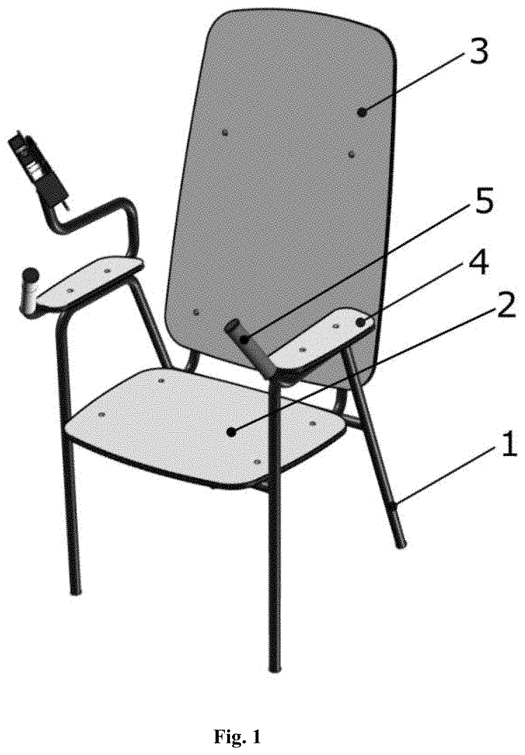

FIG. 1 illustrating an example, non-limiting embodiment of the proposed chair for measuring the ECG in the 1-lead. The chair consists of a frame (1), a seat (2), a seat back (3) and wide armrests (4), connected by assembly operations, for example, welding, gluing, riveting and others.

The frame (1) is made of a painted metal tube with a diameter of 25-32 mm and a wall thickness of 2 mm. The seat (2), the seat back (3) and armrests (4), for example, are made of chipboard, plywood or HPL plastic with a thickness of 8-25 mm. The armrests contain dry capacitive contact electrodes (5). The electrodes (5) and armrests (4) can be interconnected by one of the following assembly operations: for example, welding, gluing, as well as threaded, pinned and other connections, including combined and movable. The dry capacitive contact electrodes (5) can be made of stainless steel and have a cylindrical shape. If the electrodes are made of metal, they must be isolated from the chair frame through a dielectric spacer. The spacer can be made of any dielectric material (plastic (ABS, PVC, polyethylene), ceramics, rubber, silicone).

The advantage of dry contact electrodes is the absence of additional preparatory manipulations with them before measuring the ECG, as well as the absence of need for additional consumables, for example, electrically conductive gels and saline solutions. However, to obtain reliable ECG results using dry contact electrodes, it is necessary to provide a large contact area with the surface of the human body. The Figures show that the maximum contact area is achieved due to the implementation of the electrodes in the form of handles, which the patient can naturally, without additional effort and tension, grasp with his/her palms. FIG. 1 shows the electrodes that are made in the form of handles installed perpendicular to the armrests, and are configured in such a way that when the arms are placed on the armrests of the proposed chair, the patient can grasp the contact electrodes (5) with his/her palms, i.e. hold onto the electrodes with his/her hands, thus establishing contact of the body with the electrodes directly at the point at which it is necessary to measure the ECG in the 1-lead. At the same time, this position of the hands is the most convenient for the user and allows maintaining complete relaxation of the body, including the arms and palms of the user, due to its ergonomic position in the chair and, accordingly, reduce hand tremor for ECG recording.

The electrodes are suitable for connection with the lead cable wire. For example, when using metal handle electrodes, a brass petal can be attached from the inside by electric resistance welding, after which a lead cable is soldered to the petal. The handle electrode is a massive metal part, and it is technologically difficult to solder the lead cable wire without spoiling the appearance of the handle, since the metal effectively removes heat from the place of heating. Therefore, for example, a petal is attached by argon or electric resistance welding, and after that the cable is soldered to it.

At least a part of the chair frame can be made of hollow tubes and the wires of the cable for the discharge of the electrodes can be hidden in them in order to prevent them from being damaged by careless use. The wires are required to connect to the electrocardiograph and allow it to be placed in any convenient place for independent measurement, for example, on a pedestal next to the chair, or in a holder at the user's eye level.

The metal frame of the chair has a terminal for connecting the ground conductor of the electrocardiograph (not shown in the Figure), which reduces electrical crosstalk on the lead cable.

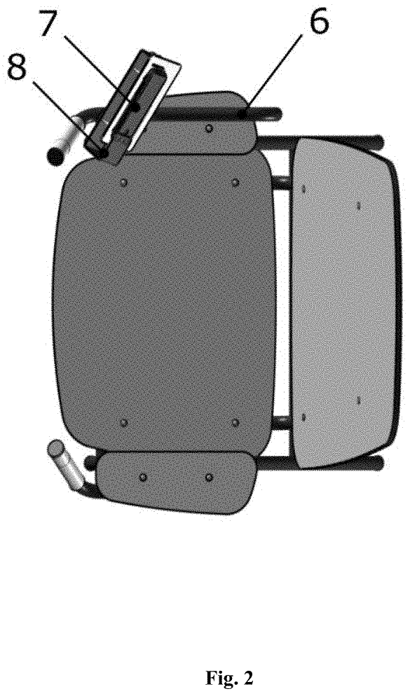

FIG. 2 shows a tube (6) for fastening the holder (7) of the electrocardiograph (8), a bent piece of a metal painted tube with a diameter of 25-32 mm, a wall thickness of 2 mm. The tube can be attached to the chair frame, for example by welding.

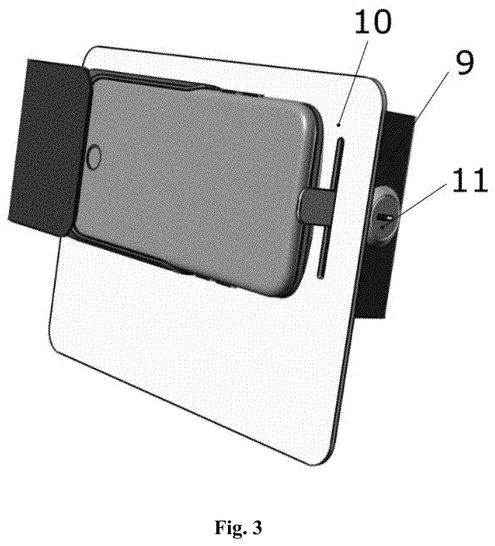

FIG. 3 shows an electrocardiograph holder (7)—an assembly unit consisting of a holder base (9) made of painted sheet metal, a mounting plate (10), for example, made of plastic, fixed through brass posts, and a lock (11). Any cardiograph suitable for recording a 1-lead electrocardiogram can be used.

To use the chair, an electrocardiograph (8) capable of recording 1-lead ECG is connected to the electrodes (5) through the cable wires (hidden inside the frame). The electrograph can be located in any convenient place for the user, for example, on a table or pedestal next to the proposed chair. According to one of the embodiments of the invention, the cardiograph is positioned at the level of the patient's eyes by fixing it in a holder (7), which is attached to the chair (as shown in FIG. 2 and FIG. 3). The user sits in the chair, independently starts the process of recording an electrocardiogram on an electrocardiograph, puts his/her arms on the armrests (4), takes the electrodes (5) by hands, takes the most comfortable body position, relaxes, waits for the end of the recording. The ergonomic shape of the device for recording the ECG in the 1-lead and the configuration of the handle electrodes make it possible to take an ECG with a minimum level of interference caused by the electrical activity of the arm muscles.

The proposed medical chair allows a seated patient to place his/her hands, including palms, in a natural position, but with maximum contact with the electrodes, thereby providing a high degree of reliability of the ECG results in the 1-lead. The results of the medical ECG in the 1-lead can diagnose arrhythmias, coronary heart disease, conduction abnormalities, atrial fibrillation, and other heart diseases.

The chair, containing the above-described handle electrodes and a cardiograph, makes it possible to record a resting electrocardiogram in the 1-lead while sitting in the most comfortable and comfortable body position completely independently by the user (patient).

The given specific examples of the implementation of the technical solution do not limit the claimed scope of claims and a specialist will see that other variants of the proposed medical chair can be executed within the scope of the independent claim.

1. Chair for measuring electrocardiogram in the first lead containing:
two dry capacitive contact electrodes, each is attached to the appropriate armrest; wherein
electrodes are made in the form of handles and configured in such a way as to enable the user to hold onto them with their palms in a sitting position; wherein
electrodes are adapted to connect them to the lead cable for connection with the electrocardiograph,
wherein electrodes are isolated from the chair frame through a dielectric spacer,
wherein the electrocardiograph holder is attached to the chair so that the electrocardiograph secured in the holder is at user eye level.

2. The chair of claim 1, wherein the electrodes are cylinder shaped.

3. The chair of claim 1, wherein the electrodes are made in the form of stainless steel handles.

4. The chair of claim 1, wherein the chair consists of a frame, seat, seat back and two armrests.

5. The chair of claim 4, wherein the frame is at least partially made of hollow tubes.

6. The chair of claim 5, wherein the lead cable wires are hidden in the frame tubes.

7. The chair of claim 4, wherein the frame is metal.

8. The chair of claim 7, wherein the metal frame has a terminal for connecting the electrocardiograph ground conductor.
